(19) United States
(12) Patent Application Publication
(10) Pub. No.: US 2010/0029544 A1
(43) Pub. Date: Feb. 4, 2010

(54) Composition

(76) Inventors: Woei Ping Cheng, Aberdeen (GB); Colin Thompson, Aberdeen (GB)

Correspondence Address: TAFT, STETTINIUS & HOLLISTER LLP, SUITE 1800, 425 WALNUT STREET, CINCINNATI, OH, 45202-3957, US

(21) Appl. No.: 12/373,210

(22) PCT Filed: Jul. 12, 2007
(86) PCT No.: PCT/GB07/02595

§ 371 Date: Jul. 20, 2009

(30) Foreign Application Priority Data
Jul. 12, 2006 (GB) 0613817.6
Jul. 29, 2006 (GB) 0615147.6

(51) Int. Cl. A61K 47/34; C08G 73/02; A61K 38/02; A61K 31/7088; A61K 38/28; A61K 38/13; A61K 31/56

(52) U.S. Cl. 514/3; 514/772.3; 528/422; 514/2; 514/44 R; 514/9; 514/169

(57) ABSTRACT

Novel polyallylamine (PAA) based graft polymers are provided, including groups such as cholesteryl, cetyl, palmitoyl, which are adapted to deliver an entity that is normally of poor solubility in an aqueous medium, said entity being such as a drug, peptide, protein, or polynucleotide that is releasably contained within the said polymer, the resulting complex being of nanoparticle range sizes with a T_{g} of less than 37° C. and deliverable in aqueous media as micelles of typically 100 to 500 nm in hydrodynamic diameter, thereby offering a delivery vehicle capable of oral or parenteral administration that protects the entity from enzymes and critical pH changes.

The present invention relates to a new polymer and the use of the polymer as a delivery system for substantially hydrophobic entities such as substantially hydrophobic drugs, proteins or peptides. Alternatively the polymer may be used in the delivery of DNA. Typically the delivery system of the present invention increases the water solubility of hydrophobic entities, and enhances the cell uptake of such entities.

Today, drug discovery strategies involving high throughout screening and combinatorial chemistry have resulted in many new entities which are substantially water-insoluble (1). It is estimated that 95% of new entities are poorly water soluble. This unfavourable physico-chemical property presents a challenge for formulators which often results in the failure of such drugs in the development stage.

Substantially water insoluble or hydrophobic drugs present a challenge to drug formulators as intravenous administration of such entities is hindered whilst the oral bioavailability of such entities is very low.

In the past, poorly water soluble drugs have been delivered through formulation systems involving organic solvents (2), low molecular weight surfactants (2) and/or extreme pH conditions or oil based formulations. Formulation systems containing surfactants such as Cremophor EL are known. Such formulation systems may cause adverse reactions, which may prevent or limit the success of the drug (3). Furthermore the ratio of drug to excipient in formulations is typically very low, and only limited amounts of drug will be solubilised within the drug delivery system. Accordingly, relatively large amounts of the drug formulation must be administered to ensure administration of the required amount of the drug. The large dosage regimes limit patient compliance limiting the therapeutic effectiveness of such drug formulations. Unpleasant and potentially dangerous side effects are often caused through the administration of hydrophobic drugs with a narrow therapeutic index using conventional formulations. Such side effects are often caused by the lack of tissue specificity of conventional formulations.

Known formulations for the delivery of substantially hydrophobic or water insoluble entities are commonly unstable and have limited shelf life. Additionally the price and availability of known delivery systems, or components thereof make the use of such delivery systems unfavourable.

The challenges associated with the administration of substantially hydrophobic entities are well known. Attempts to overcome these challenges through the use of amphiphilic polymers comprising a polyethyleneimine (PEI) backbone having pendant hydrophilic and hydrophobic groups attached thereto has been reported (WO 2004/026941). WO 2004/026941 also discloses that the PEI polymer may be used to administer a poorly water-soluble drug.

According to a first aspect of the present invention there is provided a polymer having a structure according to the following formula:

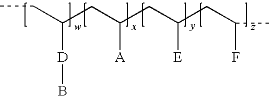

wherein:

A represents a hydrophilic group;

B represents a hydrophobic group;

D and E independently represent amine groups (in particular primary alkylamine groups);

F represents an amine group substituted with a B group; wherein the amine group is either substituted with an A group or the amine group is a quaternary ammonium moiety being substituted with four substituents;

where the molar ratio of monomeric unit Z to monomeric unit Y is 0:100 the molar ratio of monomeric unit W to monomeric unit Y is 0.01 to 100:100;

where the molar ratio of monomeric unit w to monomeric unit Y is 0:100 the molar ratio of monomeric unit Z to monomeric unit Y is 0.01 to 100:100;

the molar ratio of monomeric unit X to monomeric unit Y is 0 to 100:100.

Advantageously the polymer is a polyallylamine (PAA) polymer.

The arrangement of the monomeric units W, X, Y and z may be in any order as the hydrophilic hydrophobic attachments are random. However, preferably no more than three consecutive units should be the same.

In one embodiment the molar ratio of monomeric unit w to monomeric unit Y is 0.01 to 60:100; suitably 1 to 20:100; more suitably 1 to 10:100; advantageously 1 to 5:100.

Preferably the polymer of the present invention is amphiphilic.

Suitably the degree of hydrophilic modification is as high as possible resulting in a relatively high molar ratio of monomeric unit X to monomeric unit Y.

In one embodiment the molar ratio of monomeric unit X to monomeric unit Y is 0.01 to 100:100; typically 10 to 90:100; suitably 30 to 70:100; more suitably 40 to 60:100; advantageously 40:90.

In one embodiment the molar ratio of monomeric unit Z to monomeric unit Y is 0.01 to 60:100; suitably 1 to 20:100; more suitably 1 to 10:100; advantageously 1 to 5:100.

Suitably the polymer contains monomeric unit W and Z. Typically monomeric unit Z is present at a lower molar ratio than monomeric unit W. The molar ratio of monomeric unit Z to monomeric unit Y may be 1 to 5:100. The molar ratio of monomeric unit W to monomeric unit Y may be 10 to 200:100.

In one embodiment the polymer has a structure according to the following structure:

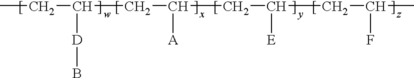

where:

D suitably represents CH₂—NH;

A suitably represents CH₂—N-hydrophilic group;

E suitably represents CH₂—NH₂;

F suitably represents

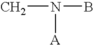

or a quaternary ammonium moiety wherein one of the substituents of the quaternary ammonium moiety is a B group.

The parent PAA compound used to make the polymer of the present invention may have an average molecular weight of about 10 to 70 kD; suitably 10 to 25 kD; advantageously approximately 15 kD.

The hydrophobic group B is suitably a hydrocarbon chain, typically having a carbon backbone of 8 to 24 carbon atoms. The hydrocarbon group may comprise alkyl and aryl components. The hydrocarbon chain may be saturated or unsaturated, and may be substituted or unsubstituted. The carbon backbone of the hydrophobic group B may be substituted with hydrocarbon groups, such as alkyl or aryl groups, in particular alkyl or aryl groups having one to ten carbon atoms. Suitably the carbon backbone of the hydrophobic group B is substituted with one or more ester, aldehyde, ketone, amine or amide groups. Alternatively or additionally the carbon backbone of the hydrophobic group may be substituted with one or more alkenyl, alkynyl, acyl, hydroxy alkyl, hydroxy acyl or sugar group.

Suitably the hydrophobic group B is an alkyl group or an acyl group, where part or all of the hydrocarbon chain of the alkyl or acyl group may be a cyclic hydrocarbon group. Suitably the alkyl group is unsaturated. Advantageously the hydrophobic group B is a cholesterol based group; typically the cholesterol based group has the structure as shown below:

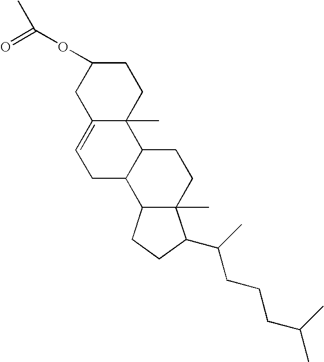

Alternatively, the hydrophobic group B may represent an alkyl chain having 15 to 20 carbon atoms such as CH₃(CH₂)₁₅.

The hydrophilic group A may suitably represent an amine. Generally the amine group is linked to the PAA carbon backbone via an alkyl group, such as a CH₂ group. The hydrophilic group A is typically a primary, secondary or tertiary amine wherein if the hydrophilic group A is a primary or secondary amine it is substituted with a hydrophilic group.

Suitably the hydrophilic group A is a primary, secondary or tertiary amine as described above substituted with one or more non-ionic group such as methyl glycolate or polyethylene glycol. Alternatively, the hydrophilic group A may be a primary, secondary or tertiary amine as described above substituted with one or more hydrogen, alkyl, alkenyl, alkynyl, aryl, acyl, hydroxy alkyl, hydroxy acyl, polyethylene glycol or sugar group. Where appropriate, the substituents listed above may be in linear, branched, substituted, unsubstituted or cyclo form.

Suitably the amine groups listed above are substituted with one or more sugar groups comprising 1 to 20 carbon atoms; more suitably 1 to 12 carbon atoms; typically 1 to 6 carbon atoms.

Alternatively the amine groups listed above may be substituted with CH₃ and H groups.

Typically the hydrophilic group represents a quaternary ammonium moiety, typically having the structure as shown below:

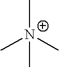

In the structure above the quaternary ammonium moiety is attached to the carbon backbone of the PAA polymer via one of the bonds shown, suitably via an alkyl group, such as CH₂. Typically the other three groups attached to the quaternary ammonium moiety are independently any of the substituents listed above; suitably H or CH₃.

As noted above, F represents an amine group substituted with a B group. Either the amine group is a quaternary ammonium moiety or the amine group is substituted with a hydrophilic group. Typically the hydrophilic group is a non-ionic group such as methyl glycolate or polyethylene glycol. Generally the amine group is linked to the PAA carbon backbone via an alkyl group, such as a CH₂ group.

Suitably the amine group of F is substituted with one or more hydrogen, alkyl, alkenyl, alkynyl, aryl, acyl, hydroxy alkyl, hydroxy acyl, polyethylene glycol or sugar group. Where appropriate, the substituents listed above may be in linear, branched, substituted, unsubstituted or cyclo form.

As noted above, D and E independently represent amine groups, suitably primary alkyl amine groups. Suitably the amine group has the structure CH₂NHR or CH₂NH₂ where R represents a substituted or unsubstituted hydrocarbon chain. R may represent hydrophobic group B.

The carbon backbone of the polymer may suitably be substituted or unsubstituted.

In one embodiment the carbon backbone of the polymer is unsubstituted. Suitably the carbon backbone of the polymer, in combination with groups D and E, consists solely of primary amines.

Typically the carbon backbone of the polymer has the structure shown below:

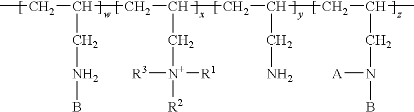

wherein A and B are as described above;

R₁, R₂ and R₃ independently represent an H, alkyl, alkenyl, alkynyl, aryl, acyl, hydroxy alkyl, hydroxy acyl, polyethylene glycol or sugar group.

Suitably the polymer is in the form of a solution, typically an aqueous solution. Alternatively the polymer is in the form of a freeze-dried composition.

Where the polymers of the present invention are amphiphilic they consist of hydrophobic and hydrophilic moieties within the same macromolecule and generally form nano self-assemblies in aqueous media. A hydrophobic core is suitably created upon contact with an aqueous media due to the aggregation of the hydrophobic moieties. The hydrophobic core can serve as a ‘microcontainer’ for molecules in particular hydrophobic molecules. These self-assemblies suitably consist of polymeric micelles, polymeric nanoparticles or polymeric vesicles.

According to a further aspect of the present invention there is provided a composition comprising the polymers of the present invention and a pharmaceutically acceptable vehicle.

Suitable pharmaceutically acceptable vehicles are well known to those skilled in the art and include aqueous and non-aqueous solutions, emulsions and suspensions. Generally the non-aqueous solutions, emulsions and suspensions include non-aqueous solvents that may be water-miscible such as propylene or polyethylene glycol, oils such as vegetable oils, or organic esters. Aqueous pharmaceutically acceptable vehicles include alcoholic/aqueous solutions, emulsions or suspensions including saline, particularly 0.9% weight/volume (w/v) saline. Typically the aqueous pharmaceutically acceptable vehicle comprises distilled water.

Preferably the composition comprises an aqueous pharmaceutically acceptable vehicle.

The composition may also comprise additives such as preservatives, antimicrobials, antioxidants, chelating agents, inert gases and the like. Suitably the ratio of polymer of the present invention:pharmaceutically acceptable vehicle by weight/volume (W/V) (g/ml) is 0.0001-100:100; typically 0.005 to 50:100; more suitably 0.001 to 10:100; advantageously 0.01-1:100.

When contacted with an aqueous media, the hydrophobic groups of the polymer described above aggregate to form hydrophobic solubilising domains within the aqueous media.

The composition may be formed by mixing the polymer described above and a pharmaceutically acceptable vehicle, suitably an aqueous pharmaceutically acceptable vehicle. Typically the composition is formed using probe sonication.

Typically the composition is stable for 2 months or more at room temperature. Preferably the composition is a substantially homogenous composition and remains homogenous upon storage for two months or more.

According to a further aspect of the present invention there is provided a delivery composition comprising the composition described above and an entity to be delivered, said entity typically having a limited solubility in an aqueous media. Typically the entity is substantially or completely water insoluble, in general the entity is substantially or completely hydrophobic. Typically the entity has an aqueous solubility of 0.001 to 0.2 mg/ml at a temperature of 15 to 25° C.

In particular the entity is a drug, peptide, protein or polymer.

Where the entity is a drug the drug is suitably a steroid such as prednisolone, oestradiol or testosterone; a drug having a multicyclic ring structure lacking polar groups such as paclitaxel; griseofulvin; amphotericin B; propofol; etoposide or an anticancer drug such as bis-naphthalimidopropyl spermine.

Where the entity is a peptide the peptide is suitably a therapeutic enzyme or hormone such as glucagon or cyclosporin; where the entity is a protein the protein is suitably a therapeutic enzyme or hormone such as insulin.

Alternatively the entity may have a limited-solubility in a non-aqueous media such as oil. Typically the entity is DNA which has a relatively high solubility in an aqueous media, but a limited non-aqueous solubility.

Where the entity is DNA, the delivery composition preferably exhibits excellent DNA binding and condensing properties.

In an aqueous media, the entity is suitably housed or encapsulated within the hydrophobic solubilising domains formed from the aggregation of the hydrophobic groups of the polymer.

Suitably the delivery composition allows delivery of the entity to a patient.

Typically the delivery composition is deliverable orally or parenterally including via a subcutaneous, intramuscular, intravenous or intrathecal route. Alternatively, the delivery composition is deliverable via a rectal, vaginal, ocular, sublingual, nasal, pulmonary or transdermal route.

The ratio of entity:polymer by weight is typically 0.001 to 100:100; suitably 1 to 100:100; more suitably 10 to 90:100; generally 30 to 70:100.

The delivery composition may suitably be in the form of a solution, tablet, suppository, capsule, powder, emulsion, gel, foam or spray.

Suitably the delivery composition is in the form of a solution; typically a transparent, translucent or opaque solution.

According to a further aspect of the present invention there is provided the delivery composition as described above for use in therapy. According to a further aspect of the present invention there is provided the use of the delivery composition as described above in the manufacture of an anaesthetic or a medicament for the treatment of an infection or a disease such as cancer, diabetes, cardiovascular disease or hereditary diseases.

According to a further aspect of the present invention there is provided a method of treatment comprising the administration of the delivery composition described above to a patient in need of treatment.

According to a further aspect of the present invention there is provided a method of increasing the solubility of an entity having limited solubility in a media comprising the steps of mixing the entity, a polymer as disclosed above and the media together to form a solution.

The media typically comprises a pharmaceutically acceptable carrier such as those listed above. Typically the media is an aqueous media. The aqueous media may be in the form of an aqueous solution, suspension or emulsion or an alcohol/aqueous solution, suspension or emulsion including saline and buffered media.

The entity having limited solubility in aqueous media is suitably a drug, polymer, peptide or protein.

Typically the entity has limited solubility in an aqueous media; typically the entity has an aqueous solubility of 0.001 mg/ml to 0.2 mg/ml at a temperature of 15 to 25° C.

Alternatively the entity may have a limited solubility in a non-aqueous media. Typically the entity is DNA.

Suitably the polymer is mixed with the media prior to mixing of the entity.

Alternatively the polymer may be mixed with the entity prior to mixing with the media.

Suitably the polymer is mixed with the entity at a ratio of 0.001 to 100:100 by weight; generally 30 to 70:100 by weight.

Advantageously the drug loading ratio of polymer:entity is 1:2 or lower. The drug solubility increases with higher drug loading concentrations. Accordingly, the drug solubility at low polymer:entity ratios is greater than the drug solubility at high polymer:entity ratios.

Higher drug loading concentrations are also associated with the achievement of optimum therapeutic effects. Typically the polymer is added to the media, suitably aqueous media, at a ratio of 0.001 to 100:1 w/v; generally at a concentration of 0.01 to 1:1 w/v. Advantageously, the solubility of the entity is increased at least 5 fold; typically at least 10 fold; suitably at least 20 fold; more suitably 24 fold or more.

According to a further aspect of the present invention there is provided a method of promoting the absorption of an entity having limited solubility in an aqueous media comprising the steps of mixing the entity, a polymer as described above and an aqueous media.

Suitably the absorption of the entity by the human or animal body is maximised.

Typically the ability of the entity to cross biological barriers, in particular cell barriers, is maximised and the uptake of the entity in vivo or in culture cells is facilitated.

The entity may suitably be a drug, protein, polymer, peptide or DNA.

As noted above, upon contact with an aqueous media the hydrophobic groups of the polymer described above aggregate to form hydrophobic solubilising domains or cores. The hydrophobic solubilising domains typically protect the entity from degradation following administration.

According to one embodiment of the present invention the entity is DNA. A complex is typically formed upon contact of the polymer described above with DNA through the electrostatic attraction between the amine groups (D and E) of the polymer and the phosphate groups of the DNA molecules. The complex is very stable and suitably protects the DNA molecules from degradation following administration, in particular through administration via injection. Typically the ability of the DNA to cross biological barriers is maximised, to allow the DNA entry into the nucleus of a cell. The cellular uptake of the DNA is therefore facilitated.

According to a further aspect of the present invention there is provided a method of producing the drug delivery composition described above comprising the steps of mixing the polymer described above, an entity to be delivered having limited solubility in a media and the media.

Typically the mixing step involves the use of probe sonication.

Suitably the polymer is mixed with an aqueous media prior to mixing of the entity.

Alternatively the polymer may be mixed with the entity prior to mixing with the aqueous media.

The structure of the drug delivery composition so formed may be analysed and verified using any suitable technique. For instance the PAA polymer in the delivery composition may be analysed and characterised using IR, (¹³C) NMR or elemental analysis.

The invention will now be described by way of example only having reference to the accompanying Figures in which:

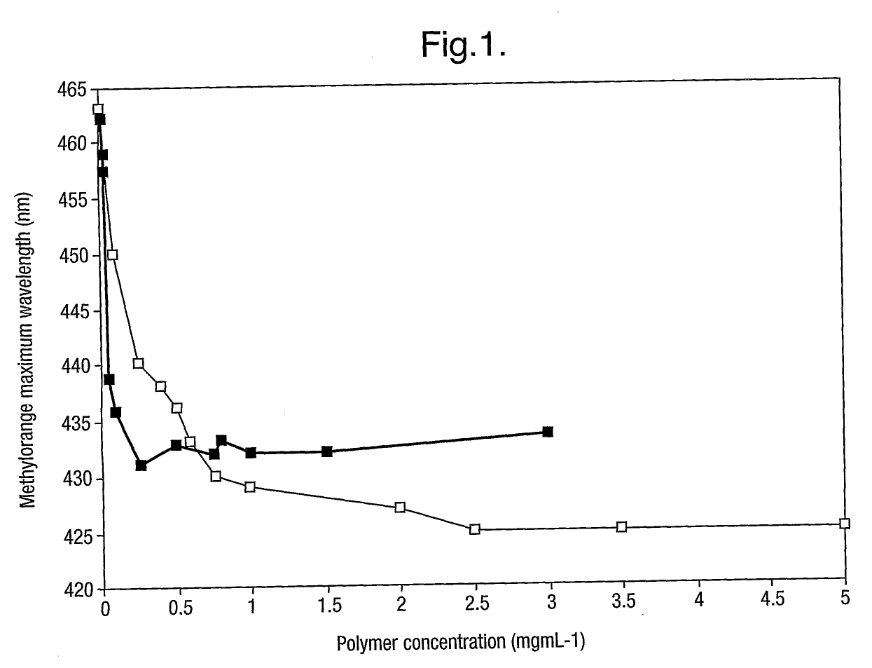

FIG. 1 shows the relationship between polymer concentration and methyl orange maximum wavelength evidencing the polymer aggregations formed through the hypsochromic shift in the methyl orange spectrum wherein the black square represents the results obtained using cholesteryl carbamate PAA (CPAA) and the white square represents the results obtained using quaternary ammonium cholesteryl carbonate PAA compound (QCPAA);

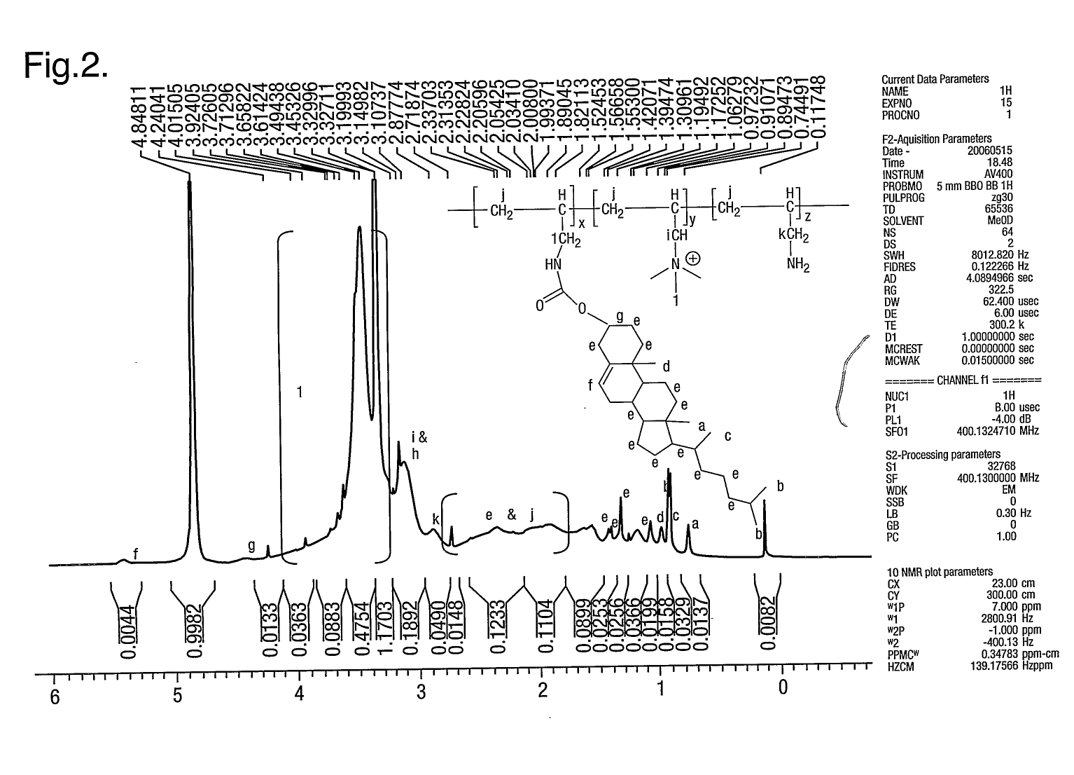

FIG. 2 shows a ¹H NMR spectrum of a quaternary ammonium cholesteryl carbonate PAA compound;

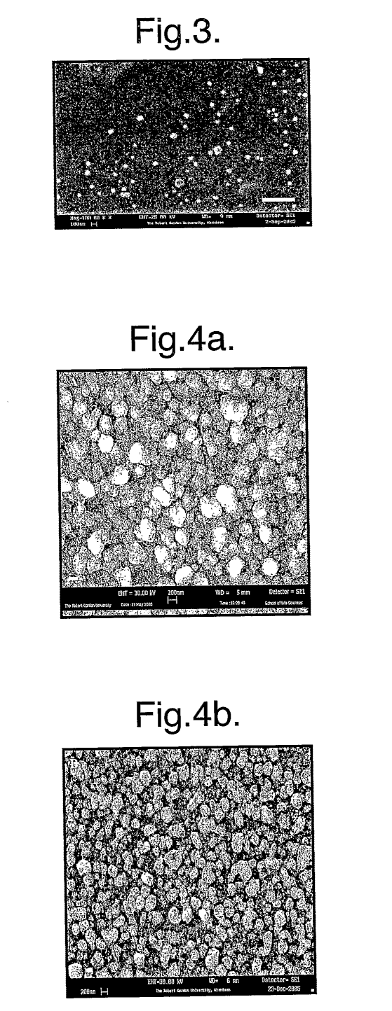

FIG. 3 shows an SEM image of a cholesteryl carbamate PAA (CPAA) compound in aqueous solution (1 mg/ml) filtered and stored for two months at room temperature;

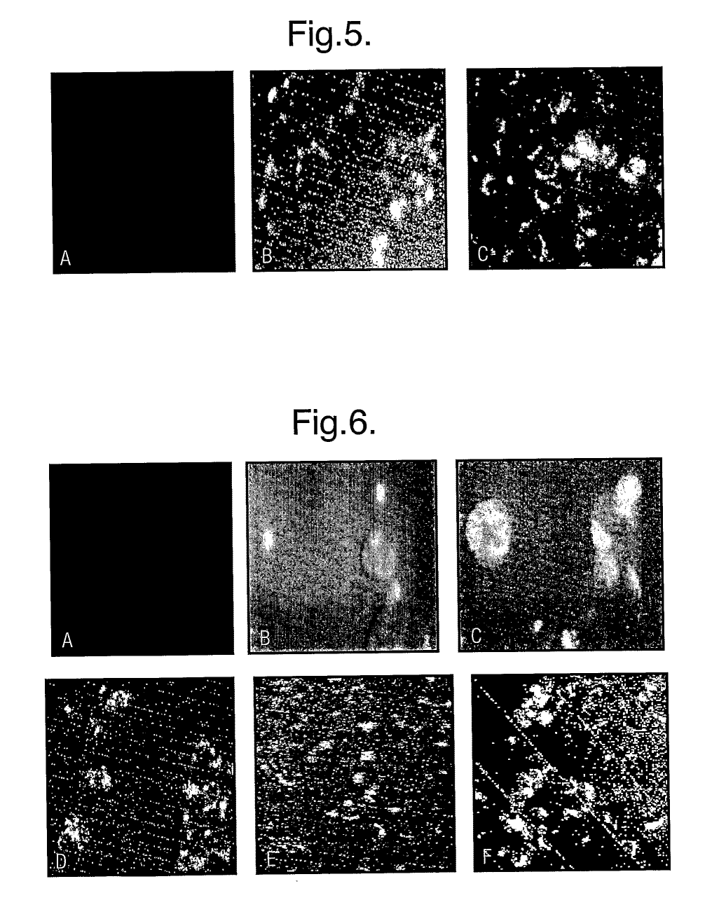

FIGS. 4A and B show an SEM image of a filtered quaternary ammonium cholesteryl carbamate PAA (QCPAA) (5 mg/ml) propofol (2.82 mg/ml) formulation and an SEM image of a filtered, freshly prepared cetyl PAA (3 mg/ml) propofol (1.63 mg/ml) formulation respectively;

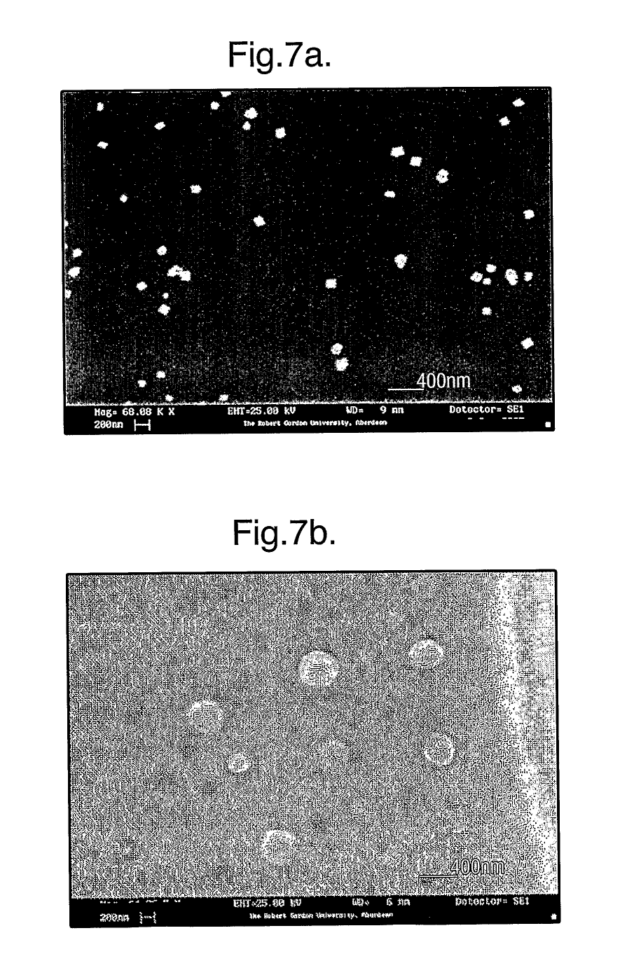

FIGS. 5 A, B and C show Caco-2 cells incubated with PK4 (20 μM) at 37° C. after 1 hour, 4.5 hours and 24 hours respectively;

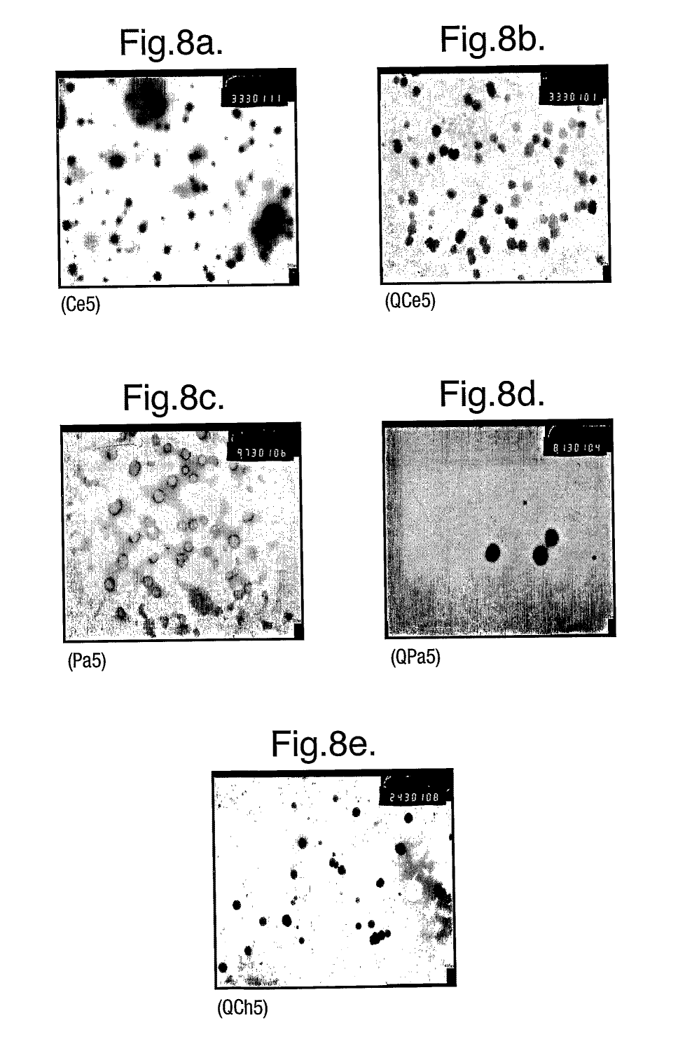

FIG. 6 shows Caco-2 cells incubated at 37° C. with QCPAA (0.1 μg/ml) PK4 (20 μM) solution, wherein A shows the control medium and Figures B to F show incubation after 30 minutes, 1 hour, 2 hours, 3 hours and 24 hours respectively;

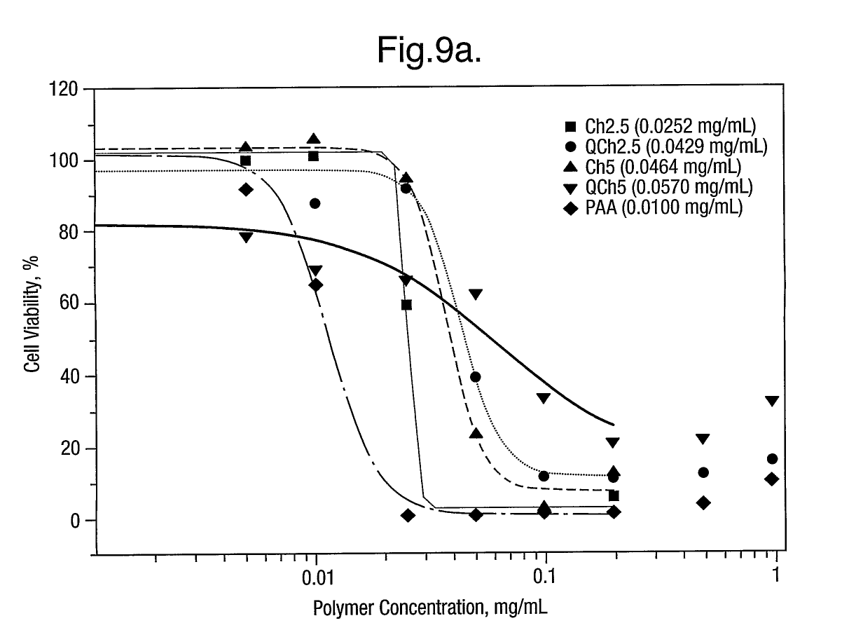

FIGS. 7 (a) and (b) show SEM micrographs of amphiphilic copolymer micelles and quaternised copolymer micelles;

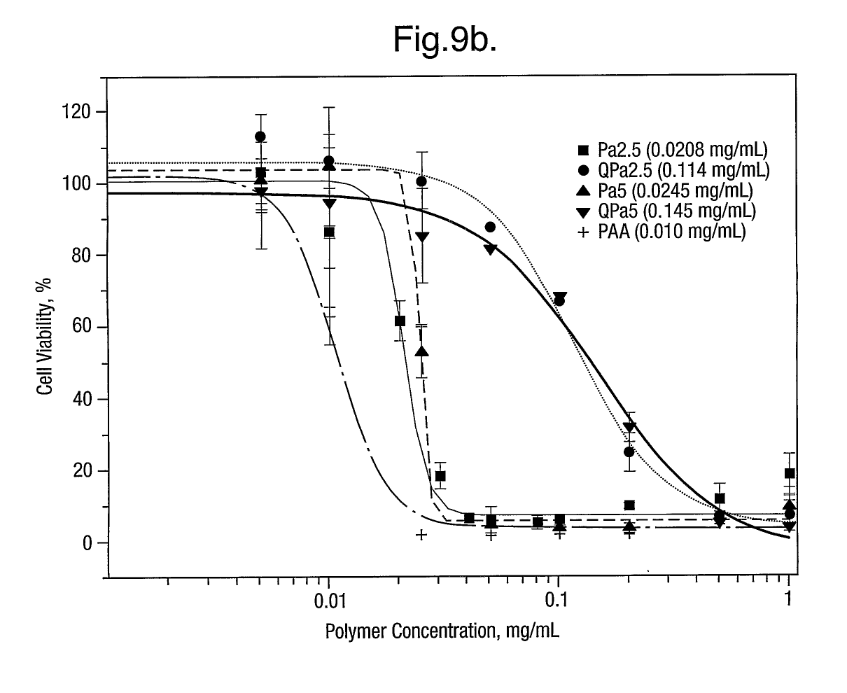

FIGS. 8 A, B, C, D and E, show TEM imaging of different graft polymer solutions in water at 1 mg/ml, after 2 weeks in solution and demonstrating stable retention of spherical structure over time at room temperature;

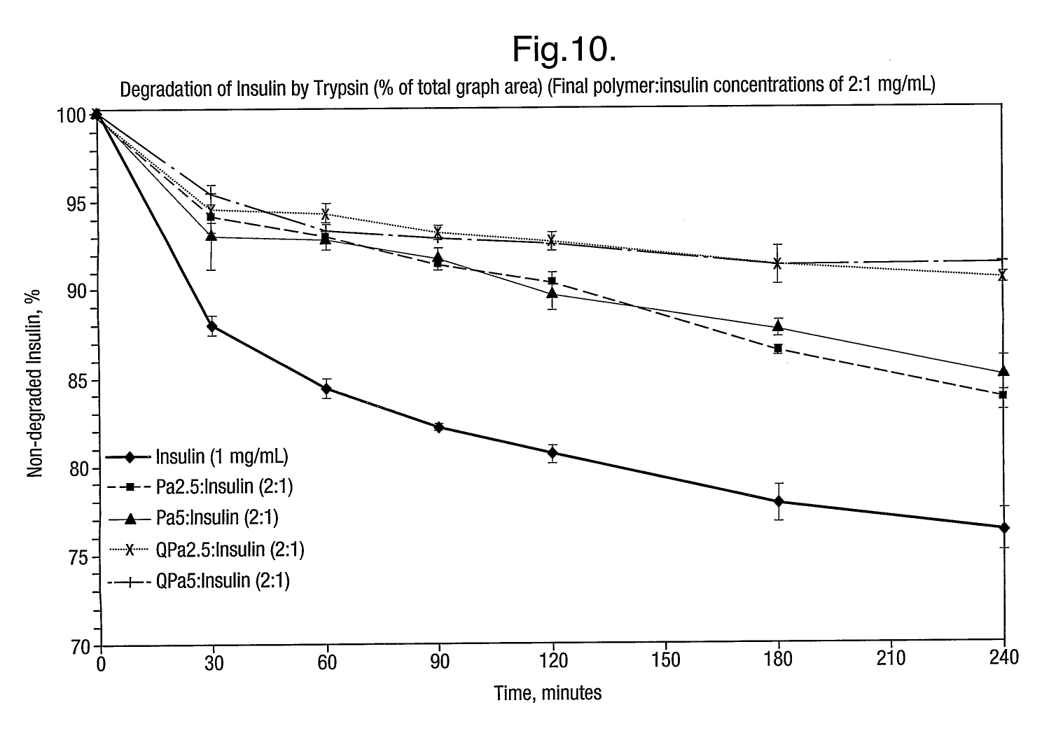

FIGS. 9a and 9b show data from a colorimetric assay (MTT) of Caco-2 cells to determine IC50 values for cholesteryl and palmitoyl graft copolymers;

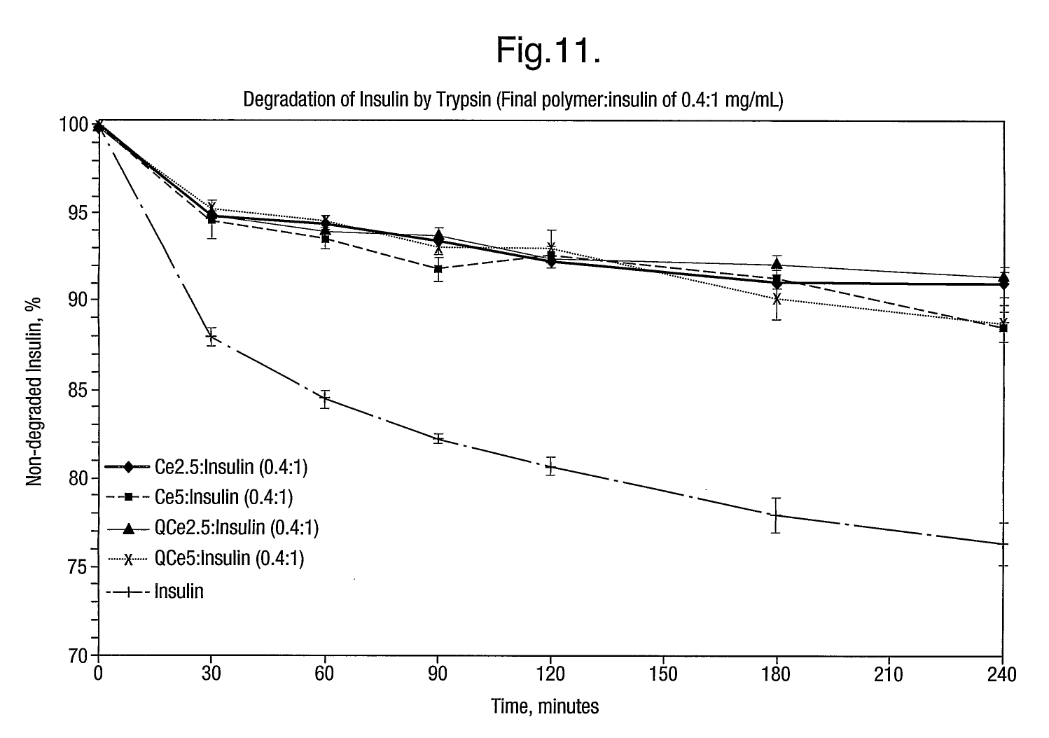

FIG. 10 shows data on degradation of insulin by trypsin comparing palmitoyl-graft polymer complexes with control uncomplexed insulin;

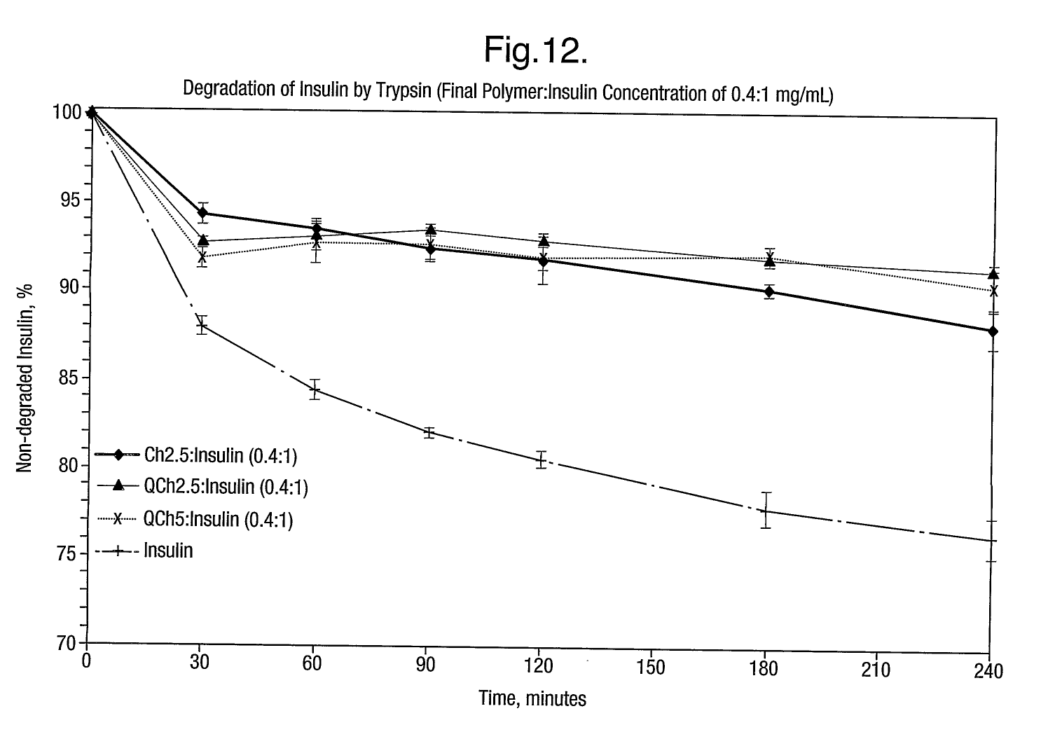

FIG. 11 shows data on degradation of insulin by trypsin comparing cetyl-graft polymer complexes with control uncomplexed insulin;

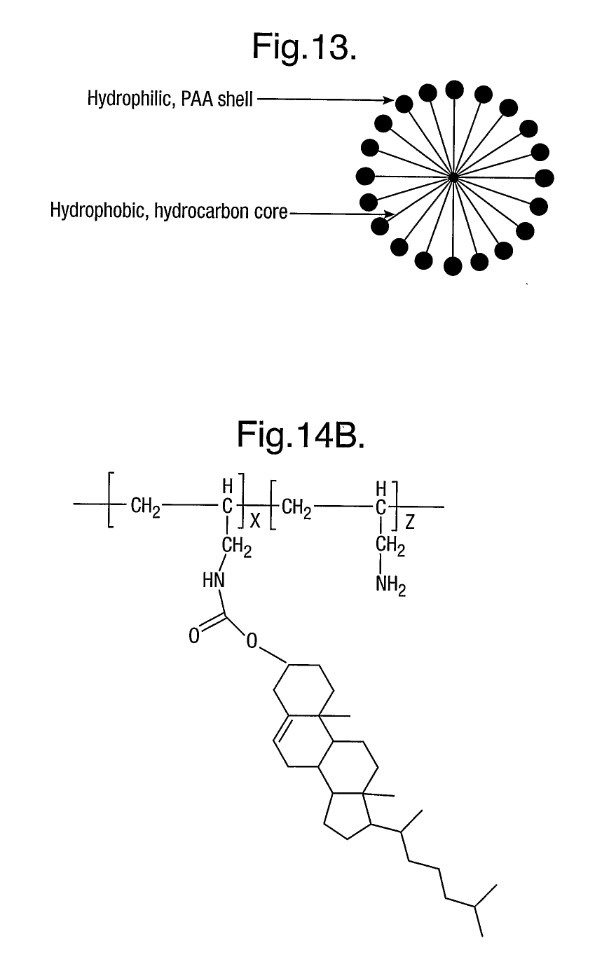

FIG. 12 shows data on degradation of insulin by trypsin comparing cholesteryl-graft polymer complexes with control uncomplexed insulin;

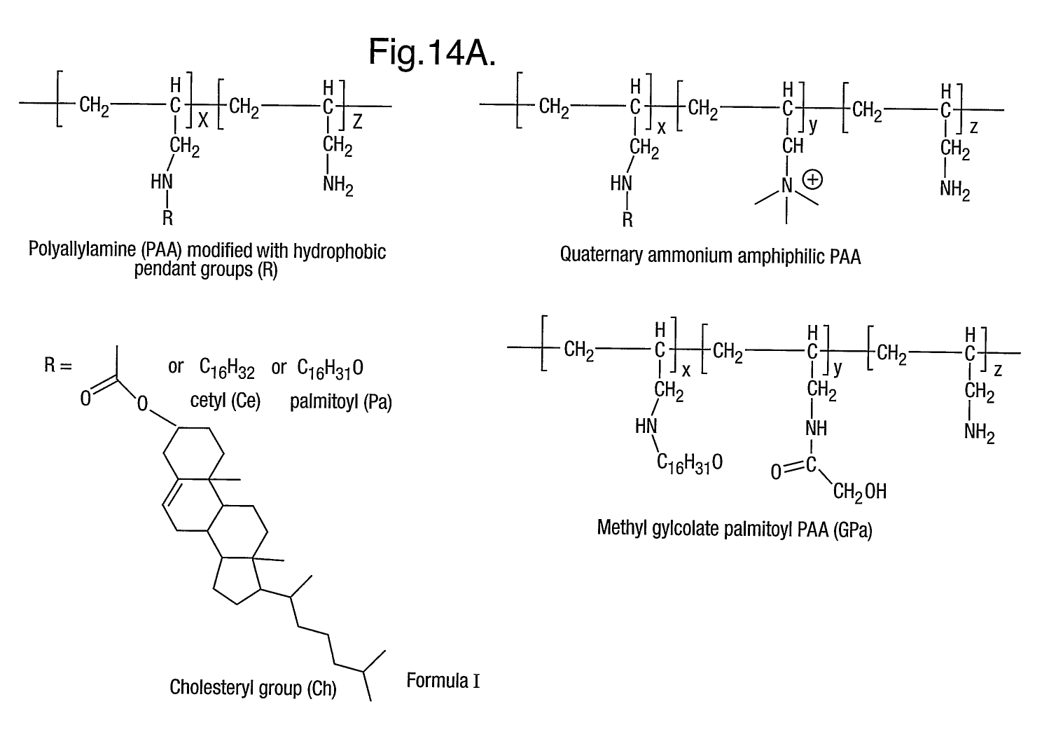

FIG. 13 is a schematic representation of modified PAA polymeric species of the invention as rearranged in a micellar form with a hydrophobic core and hydrophilic shell;

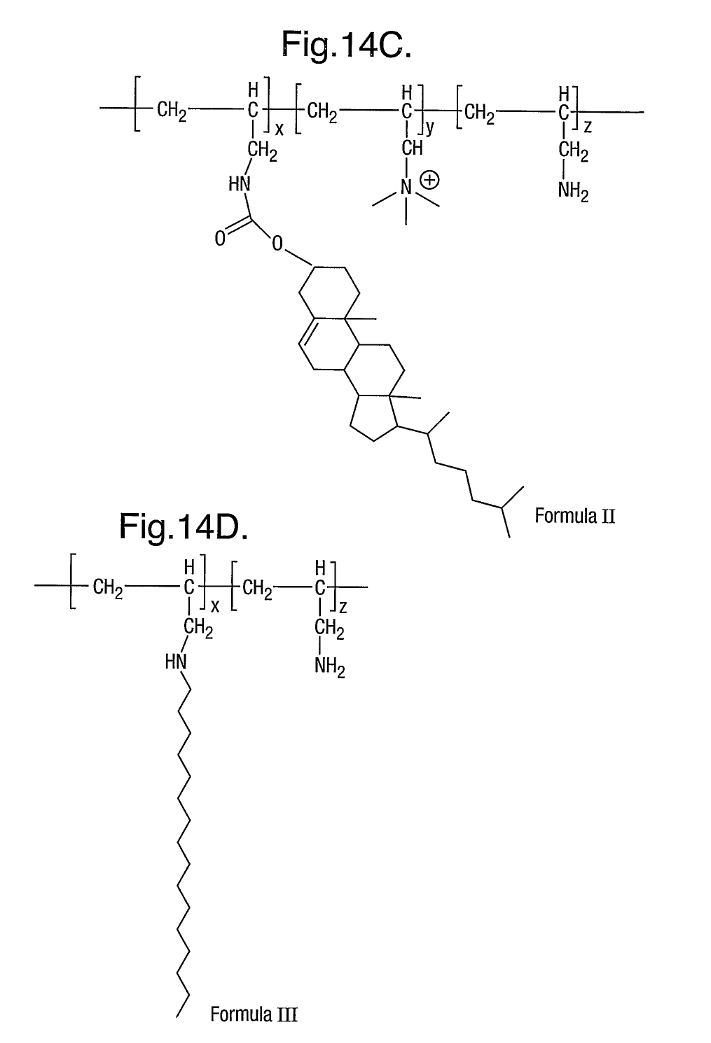

FIG. 14 (A-D) shows structural formulae referred to in the specification, particularly hydrophobic group (R)-modified polyallylamine (PAA), where R may be cholesterol, cetyl or palmitoyl, quaternised PAA, and methyl glycolate palmitoyl PAA (GPa).

The invention will now be further described by way of the following illustrative examples.

### EXAMPLE 1

### Polymer Synthesis

10 g of 15 kDa PAA.HCl was dissolved in distilled water and approximately 8 g of NaOH pellets were added until an alkaline pH (˜13) was reached. The solution was then subjected to exhaustive dialysis (Visking tubing, molecular weight cut off=12000-14000) against 5 L of distilled water for 24 h with six changes. The dialysate was then freeze-dried. The freeze-dried material consists of PAA free base.

This PAA free base will then be used as the starting polymer for all amphiphilic polymer synthesis.

### EXAMPLE 2

### Synthesis of Cholesteryl carbamate PAA (CPAA)

15 kDa (1.5 g) was dissolved in 30 ml of chloroform and methanol (1:1) containing 0.25 ml of triethylamine. Cholesteryl chloroformate (0.5 g) dissolved in chloroform and methanol (1:1) solution (20 ml) was added drop wise to the solution containing PAA. The reaction mixture was then stirred for 24 h and the solvent was evaporated. The product was washed three times with 100 ml diethyl ether, dissolved in distilled water and then dialysed exhaustively against distilled water as described above. The dialysed product (CPAA) was freeze-dried and presented as a cotton-like solid.

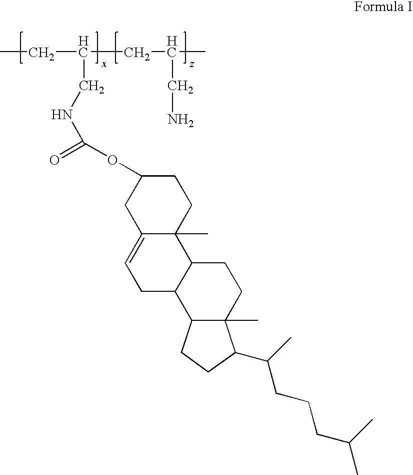

### EXAMPLE 3

### Synthesis of Quaternary Ammonium Cholesteryl Carbamate PAA (QCPAA)

300 mg of CPAA was dissolved in 10 ml of methanol overnight at room temperature. Methyl iodide (1.3 ml), sodium hydroxide (112 mg), and sodium iodide (128 mg) were then added to the mixture and the solution was stirred under a stream of nitrogen for 3 h at 36° C. The resulting solution was then added drop-wise to diethyl ether (400 ml) and the precipitate formed was allowed to settle overnight. The solution was poured off and the precipitate was then dissolved in a water/alcohol mixture (1:1 ratio-100 ml each) and subsequently subjected to exhaustive dialysis. An ion exchange procedure was carried out by packing a column (1×6 cm) with one volume of Amberlite-96 resin (30 ml). The column was subsequently-washed with 100 ml of 1M HCl. Distilled water was passed through the column until a neutral pH was obtained. The dialysed product was passed through the column and the elute was freeze-dried to give a ‘cotton-like’ solid.

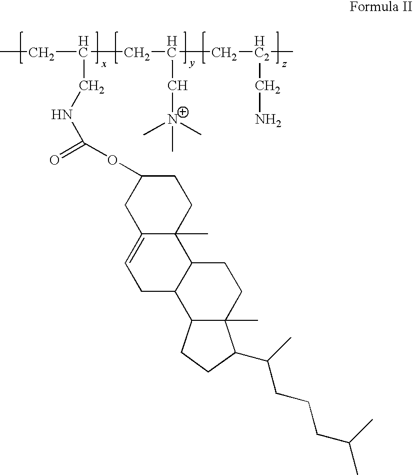

### EXAMPLE 4

### Synthesis of Cetyl PAA (CePAA)

PAA (1 g) and cetyl bromide (2.7 ml) were refluxed in 50 ml of chloroform, methanol (1:1) for 48 h. Methanol (20 ml) containing NaOH (1 g) was added to the above solution and the resulting solution was refluxed for another 24 h. Sodium bromide was filtered and the solvent was subsequently evaporated at 50° C. The product was then washed three times with 100 ml diethyl ether, dissolved in distilled water, dialysed exhaustively against distilled water and freeze-dried as detailed above.

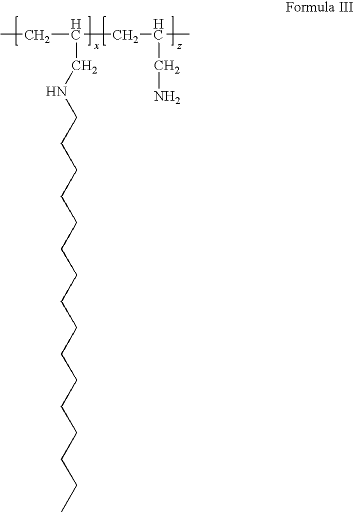

### EXAMPLE 5

### Synthesis of Palmitoyl PAA

PAA (2 g) and 2.352 g of sodium hydrogen carbonate were dissolved in 100 ml of distilled water with stirring. Palmitic acid-N-hydroxysuccinimide ester was dissolved in 100 ml of ethanol and added drop-wise over 1 hour to the PAA solution. The reaction was carried out at 25° C. over 72 hours with stirring at 500 rpm. The solvent was then evaporated and the residue was dissolved in 50 ml of water, purified and freeze dried.

Polymer Structural Characterisation

IR spectrum of CPAA was obtained using Nicolet-380 FITR spectrometer (Thermo Electron Corporation, UK) and ¹³CNMR spectroscopy (Bruker AMX 400 MHz) was performed on QCPAA dissolved in deuterated methanol. Elemental analysis was conducted on all polymers using a Perkin Elmer 2400 Analyser.

Polymer structures were confirmed by FTIR, NMR and the level of polymer modification confirmed by elemental analysis. The bands at 1696 and 1561 cm⁻¹ of the FTIR spectrum confirmed the presence of amide bonds in CPAA indicating the attachment of cholesteryl moieties onto the PAA backbone. The synthesis of QCPAA was confirmed by ¹HNMR and ¹³CNMR. ¹HNMR assignments are shown in FIG. 1. ¹³CNMR assignments are as follows δ42.98=(CH₃)₂N (PAA), δ51.49−52.48=CH₂N, CH₂N⁺ (PAA). The mole % modification values for 3 polymers were obtained from the elemental analysis data, which are shown in Table 1. The values of amine conversion to quaternary ammonium moieties were approximate. An assumption was made that quaternisation resulted in quaternary amines although inevitably, some will still exist as tertiary amines.

TABLE 1
elemental analysis data

| | Mole % | | Mole % |
|---|---|---|---|
| | Cholesterol | Mole % | quaternisation |
| | attachment per | cetylation per | per PAA |
| Sample | PAA monomer | PAA monomer | monomer |
| CPAA | 5 | — | — |
| QCPAA | 5 | — | 45 |
| CePAA | — | 8 | — |

Polymer Aggregation Studies

The formation of polymer aggregations in aqueous media was studied using a UV visible hydrophobic probe, methyl orange. A 25 μM methyl orange solution in borate buffer (0.02M, pH 9.4) was prepared. This solution was then used as a diluent in preparing various polymer concentrations by probe sonication (Soniprep Instrument, UK) for 5 minutes at 75% of maximum output. The presence of polymeric hydrophobic solubilising domains in aqueous media was determined by observing the hypsochromic shift in the λₘₐₓ of methyl orange absorption spectra (300 nm-600 nm).

The hydrodynamic size of polymer aggregates in aqueous solutions was measured using photon correlation spectroscopy (Malvern Zetasizer 3000HS_{A}, Malvern Instruments, U.K). The polymer solutions (1 mgml⁻¹) were filtered with 0.45 μm filters before size measurements to remove dust particles. A drop of the filtered polymer solution was placed on a copper grid and air-dried. The sample was then coated with gold/palladium using a Polaron SC7640 Plasma Magnetron Sputter (Quorum Technologies, UK) and visualised using a LEO S430 scanning electron microscopy (Quorum Technologies, UK) at 25 kV. The SEM images are shown in FIG. 3.

CPAA at 1 mgml⁻¹ produced a translucent solution on probe sonication in water and the size of polymer aggregates was 252 nm. The presence of quaternary ammonium moieties resulted in clear, micellar aggregates with the hydrodynamic size of 164 nm. An increase in particle size was observed at the QCPAA concentration 5 mgml⁻¹, which indicates a possible growth in the particle association number at higher concentrations. The particle size of CePAA aggregates at 1 mgml⁻¹ was smaller than CPAA. It is thought that this phenomenon was possibly due to the formation of smaller hydrophobic cores, which could be the result of the flexibility of the cetyl chains in comparison to the bulky, rigid cholesterol moieties.

The size of filtered CPAA nanoparticles imaged by SEM (FIG. 3) was slightly smaller than the size measured by PCS. They were approximately 100 nm in size and remained stable for more than 2 months at room temperature.

TABLE 2
Size measurement of polymer aggregates in aqueous
solution after 0.45 μm filtration

| Polymer in | Storage at room temperature for 3 days | | |
|---|---|---|---|
| aqueous | | particle size (nm) | Poly-dispersity |
| solution | Appearance | (mean ± s.d., n = 3) | (mean ± s.d, n = 3) |
| CPAA | Translucent | 252 ± 1 | 0.65 ± 0.01 |
| (1 mgmL⁻¹) | | | |
| QCPAA | Clear | 164 ± 6 | 0.60 ± 0.09 |
| (1 mgmL⁻¹) | | | |
| QCPAA | Clear | 225 ± 7 | 0.53 ± 0.09 |
| (5 mgmL⁻¹) | | | |
| CePAA | Translucent | 102 ± 1 | 0.52 ± 0.01 |
| (1 mgmL⁻¹) | to clear | | |

Methyl orange is an hydrophobic probe. When it is solubilised within the hydrophobic core of micelles, the λₘₐₓ of methyl orange undergoes a hypsochromic shift. The hypsochromic shift of methyl orange from 465 nm upon contact with the nanoaggregate of the present invention evidences that these novel nanoaggregates were able to form hydrophobic domains upon aggregation of the hydrophobic groups in aqueous medium (FIG. 1). The inflection point of the curve indicates the critical aggregation concentration (CAC), which occurred at 0.15 mgml⁻¹ for QCPAA and 1 mgml⁻¹ for CPAA (FIG. 1). The presence of quaternary ammonium moieties lowered the CAC, possibly due to the introduction of adequate hydrophilic character to maintain the stability of the aggregates in aqueous solution. The polarity of the hydrophobic core, as indicated by the extent of the methyl orange hypsochromic shift when the CAC is established, is dependant on the hydrophobicity of the polymer. CPAA that had not been quaternised caused a stronger hypsochromic shift in the methyl orange spectrum, indicating the formation of less polar aggregates (FIG. 1).

Toxicity evaluation of polymers of the above type was carried out using the MTT assay (a standard calorimetric assay) which is indicative of cell viability by observing a colour change using a yellow tetrazole based dye which is reduced to a purple formazon in the mitochondria of living cells. Results for cholesteryl graft polymers and palmitoyl graft polymers (without pre-filtration) are reported in Table 3 (cholesteryl) and Table 4 (palmitoyl) below:

TABLE 3
MTT data for cholesterol graft copolymers (n = 3).

| | IC₅₀,* |
|---|---|
| Polymer | mg/mL |
| PAA | 0.0100 |
| Ch2.5 | 0.0252 |
| Ch5 | 0.0464 |
| QCh2.5 | 0.0429 |
| QCh5 | 0.0570 |

TABLE 4
MTT data for palmitoyl copolymers (n = 3).

| | IC₅₀, |
|---|---|
| Polymer | mg/mL |
| PAA | 0.0100 |
| Pa2.5 | 0.0208 |
| Pa5 | 0.0245 |
| QPa2.5 | 0.1140 |
| QPa5 | 0.1450 |

*IC50 Median Inhibition Concentration (concentration that reduces the effect by 50%)

MTT data for cholesteryl graft copolymers suggests that they are less biocompatible than palmitoyl graft copolymers. However, as is also observed with palmitoyl copolymers, quaternisation has increased the IC₅₀ value of the polymers. The drop in IC₅₀ compared to palmitoyl could be accounted for by the increased hydrophobicity and therefore poor aqueous solubility of the cholesteryl copolymers compared to palmitoyl. However it should be emphasised that all cholesteryl copolymers, as with palmitoyl, have a higher IC₅₀ than PAA.

The following examples illustrate the use of polymers as discussed above in reversible encapsulation of drugs (drug-loading) by forming a protective assembly around the drug molecule for use of the resulting complex as a delivery vehicle in a physiological system.

### EXAMPLE 6

### Loading of Griseofulvin

QCPAA polymer aggregates were prepared by probe sonication in distilled water. Drug loading was then achieved by probe sonication of the drugs in the presence of polymer aggregates in distilled water. Polymer, drug solutions with varying polymer: drug weight ratios were prepared. The solutions were then filtered (0.45 μm) and the filtrates were dissolved in methanol and subsequently analysed using an ultraviolet-visible spectrophotometer at the wavelength of 292 nm. The aqueous solubility of griseofulvin was determined by sonicating excess drugs in distilled water. The solution was then filtered and analysed as described earlier. A griseofulvin calibration curve was prepared using various standard solutions (1-30 μgml⁻¹, R²=0.993). The best formulation was sized using photon correlation spectroscopy (PCS).

All filtered QCPAA polymer, drug solutions gave rise to clear, micellar liquids. At low polymer concentration (1 mgml⁻¹), the aqueous solubility of griseofulvin did not increase compared to the intrinsic solubility (48.3±1.0 μgml⁻¹). As anticipated, at higher polymer concentration (3 mgml⁻¹), an increase in the drug solubility was observed at all polymer, drug weight ratios indicating the incorporation of griseofulvin in the polymer aggregates. The drug loading concentration is also a paramount factor in determining the level of drug incorporation, where an increase in drug recovery was detected at higher drug loading concentrations. At polymer concentration 3 mgml⁻¹, the maximum drug encapsulation was achieved at polymer, drug weight ratio of 1:2, which produced a clear micellar solution with a particle size of 124 nm. At this ratio, QCPAA 3 mgml⁻¹ is able to improve the solubility of griseofulvin up to 5-fold.

### EXAMPLE 7

### Loading of Propofol

The polymer, drug weight ratios of 1:2 and 1:1 were prepared using QCPAA (5 mgml⁻¹) or CePAA (3 mgml⁻¹). Drug loading was carried out as described above. To determine the concentration of encapsulated drug, the solutions were filtered (0.45 μm) and the filtrates were dissolved in methanol and subsequently analysed using an ultraviolet-visible spectrophotometer at the wavelength of 272 nm. A propofol calibration graph was prepared using various standard solutions (0.012-0.16 mgml⁻¹, R²=0.998). The filtered formulations were sized using photon correlation spectroscopy and imaged by scanning electron microscopy (SEM).

Both filtered QCPAA, propofol formulations were homogenously opaque with a hydrodynamic size of 762 nm while filtered CePAA, propofol formulations gave rise to translucent liquids with a much smaller particle size. The SEM image revealed that the particle size of filtered QCPAA (5 mgml⁻¹), propofol (2.82 mgml⁻¹) formulation ranged from approximately 200 nm to 500 nm. A smaller particle D size was observed for CePAA (3 mgml⁻¹), propofol (1.64 mgml⁻¹) formulation, which varied from 100 nm to 400 nm (FIG. 4). The intrinsic solubility of propofol was 0.112 mgml⁻¹. A similar trend was observed for both QCPAA and CePAA where propofol solubilisation was nearly doubled when the drug loading was increased two-fold. At the polymer, drug weight ratio of 1:2, QCPAA (5 mgml⁻¹) was able to encapsulate 2.82 mgml⁻¹ propofol, while CePAA at a lower concentration (3 mgml⁻¹) was able to solubilise 1.63 mgml⁻¹ respectively. This revealed that propofol 7 solubility could be augmented to 24-fold and 16-fold by QCPAA and CePAA respectively.

### EXAMPLE 8

### Insulin

A novel amphiphilic comb-shaped polymer was synthesised as in Example 1 above, by grafting PAA (15 KDa) with 5% mole cholesteryl chloroformate (Ch), at molar ratios of 1:0.05 (Ch₅) and 1:0.025 (Ch_{2.5}). Polymer structures were characterised using elemental analysis.

Self-assemblies were formed by probe sonicating the polymers in water before particle size analysis (Zetasizer Nano-ZS, Malvern Instruments, UK) and surface tension measurement (Torsion Balance, OS, White Electrical Instrument Co., UK) were conducted.

A hydrophobic probe, methyl orange was used to study the presence of polymer hydrophobic domains in aqueous solution by monitoring the hypsochromic shift in the methyl orange absorption spectra.

Elemental analysis confirmed the synthesis of Ch-PAA and the results correlated well with the predicted hydrophobic modification (5.0% mole for Ch-PAA). The size of nano-aggregates formed in aqueous solutions at polymer concentration 1 mgml⁻¹ was 183 nm for Ch-PAA. The critical association concentration (CAC) for Ch-PAA was calculated as 0.09 mgml⁻¹. Hypsochromic shift in the methyl orange absorption spectra was observed at 0.05 mgml⁻¹ for Ch-PAA.

Such a novel amphiphilic graft polymer was subsequently quaternised (Q) with a methyl iodide as quaternisation reagent.

The polymer and insulin solutions were made up in Tris buffer (pH 7.4) and a fixed insulin concentration (0.3 mgml⁻¹) was used. Complexation was carried out at polymer: insulin mass ratios of 2:1, 1:1 and 1:3. Complexes were left at 20° C. for 2 hours before particle size, zeta potential (Zetasizer Nano-ZS, Malvern Instruments, UK) and complexation efficiency (Luminescence spectrometer LS55, Perkin Elmer, UK) were determined. The concentration of insulin in the nano-complexes was calculated from a calibration graph (0.05-0.3 mgml⁻¹, R²=0.98).

Insulin is negatively charged at pH7.4 and since all formulations have positive zeta potentials, this indicates the complexation was successful with CE up to 103% for QAm_{2.5}. From the data it appears that non-quaternised and quaternised polymers exhibit different trends. Higher levels of complexation were achieved at lower non-quaternised polymer concentrations. However CE increased when higher concentrations of quaternised polymer were used, possibly due to an increased level of positive charge of polymers forming compact nano-complexes with insulin.

These novel amphiphilic polymers are able to form nano-complexes with insulin and thus offer a new group of insulin delivery systems.

Novel comb-shaped amphiphilic polymers were successfully synthesised and they formed nano self-assemblies in aqueous environments with potential of encapsulating hydrophobic drugs. Different grafted hydrophobic groups seem to have an impact on the properties of the self-assemblies in aqueous solution.

FIGS. 7A-E show the architecture of different graft copolymer solutions in water at 1 mg/ml. These images were taken after 2 weeks in solution demonstrating that the copolymers retained a spherical structure after that time at room temperature. This is important as it demonstrates their stability over time.

Both cetyl (Ce5) solutions appeared to be nanoparticles. This would suggest that the interior of the nanoparticles was highly hydrophobic such that the staining dye used for TEM could not penetrate into it.

Palmitoyl (Pa5) copolymer solutions were different to cetyl, even though the only difference in their structures was the extra oxygen molecule (producing a carbonyl bond) in each palmitoyl graft. Non-quaternised-palmitoyl appeared to form a bilayer in solution. This suggests that Pa5 has a less hydrophobic interior than Ce5 even with this small change in structure. Quaternised palmitoyl (QPa) appears to be nanoparticles even though the process of quaternisation should not affect the hydrophobic interactions present. It would appear that quaternisation of the palmitoyl has increased hydrophobic interactions within the core of the particles.

Quaternised cholesterol (QCh5) polymers are also nanoparticles. This was expected as cholesteryl copolymers are more hydrophobic than either palmitoyl or cetyl given their increased chain lengths.

The novelty of these graft copolymers is exemplified by the fact that they are able to form stable nanoparticles/bilayer structures which are stable for over 2 weeks which has only been accomplished before at much higher levels of palmitoyl and cetyl grafting (up to 10 times the level of grafting achieved here) or when these copolymer solutions have been supplemented with free cholesterol (which acted as a membrane stabilising agent).

FIG. 8 demonstrates that all palmitoyl complexes provide some protection from trypsin degradation over 4 hours. However quaternised palmitoyl complexes had a higher level of non-degraded insulin after 4 hours than non-quaternised complexes. This was unexpected given their respective morphologies (see TEM). Insulin appeared to aggregate on the surface on the quaternised palmitoyl particles whereas it appeared to be in the core of the non-quaternised particles. It was expected that insulin in the core would have a greater degree of protection. It may be that quaternisation has some effect on trypsin activity in that the nature of the surface charge of the quaternised particles prevented trypsin acting on its target sites more so than entrapment in the core of the non-quaternised particles.

FIG. 9 also shows that complexation with cetyl copolymers protected insulin. The levels of non-degraded insulin are similar to that of palmitoyl even though the ratio of polymer:insulin is much lower (0.4:1 compared to 2:1 for palmitoyl). The change in ratio between palmitoyl and cetyl was due to the % transmittance and size data discussed earlier in this document. This data showed that the optimal ratio of cetyl (and cholesteryl):insulin was 0.4:1 mg/mL. However this time it seems that the level of cetyl grafting (marginally) affected the extent of insulin degradation. 2.5% grafts (quaternised and non-quaternised) had slightly higher levels of non-degraded insulin after 4 hours than copolymers with 5% cetyl grafts. It may be that 2.5% grafts produced more stable complexes with insulin. However all complexes appeared translucent (including palmitoyl complexes) with no sign of precipitation (which would indicate instability) and any differences between 2.5% and 5% were almost within the standard deviation of the samples.

FIG. 10 indicates that cholesteryl complexes protected insulin from trypsin degradation. The values found were in line with those found with cetyl and palmitoyl copolymer complexes. Ch5 has not yet been tested for its protective effect but, given the data above, it would not be unreasonable to assume that the values for any Ch5-insulin complexes would be similar to other cholesteryl copolymer complexes.

Overall these copolymers have been shown to produce stable complexes which protect insulin from trypsin degradation which will be important if they are to be delivered orally. Trypsin (along with chymotrypsin and pepsin) is one of the major enzymatic degraders of insulin when it is administered orally by itself. Therefore protection from degradation is vital to produce an efficacious delivery of insulin to the circulation. The problem of enzymatic degradation could also be overcome by co-administration with enzyme inhibitors, but this is not practical or advisable for patient health as they will also prevent breakdown of food as well as insulin leading to malabsorption of essential nutrients. Therefore complexing insulin with these polymers may provide a way of protecting insulin while maintaining normal digestive activity in patients.

Generally, the suitability of the graft polymeric systems described herein can be evaluated by those skilled in pharmaceutical practice and drug formulation by considering a number of factors which include the following:
The level of hydrophobic grafting;
Presence of quaternary ammonium moieties;
Ratios of polymer to active ingredient (entity to be encapsulated);
Initial concentration of active ingredient.

The following Table 5 reports results on complexation efficiency and Zeta potential, based upon work evaluating a palmitoyl polyallylamine system wherein the complex is loaded with insulin as representative of a typical active ingredient to be delivered for a therapeutic purpose.

TABLE 5
Properties of palmitoyl-PAA complexes (initial insulin concentration
of 0.3 mg/mL). Insulin concentration after complexation determined
using a stand alone fluorimeter.

| | Initial | | | | | | |
|---|---|---|---|---|---|---|---|
| | Polymer | | | | | Insulin | |
| | concentration, | | Diameter, | | Zeta | concentration | Complexation |
| [symbol] ample | mg/ml | Form*: | nm | PDI | mV | mg/mL | efficency/% |
| [symbol] AA | 0.1 | Clr/Trs | 201 (16.6) | 0.242 | 30.3 | 0.138 | 94.2 (3.05) |
| | | | | (0.018) | (1.50) | (0.004) | |
| | 0.3 | Trs/Tbd | 573 (19.9) | 0.356 | 31.9 | 0.139 | 94.5 (1.40) |
| | | | | (0.057) | (2.34) | (0.002) | |
| | 0.6 | Tbd | 2910 (569) | 0.508 | 34.9 | 0.139 | 94.7 (1.55) |
| | | | | (0.303) | (3.03) | (0.002) | |
| [symbol] a2.5 | 0.1 | Trs | 190 (117) | 0.367 | 34.2 | 0.1315 | 93.85 (0.49) |
| | | | | (0.125) | (1.39) | (0.00) | |
| | 0.3 | Trs | 144 (3.98) | 0.258 | 35.4 | 0.118 | 84.25 (2.05) |
| | | | | (0.008) | (1.08) | (0.020) | |
| | 0.6 | Clr | 162 (1.96) | 0.278 | 35.8 | 0.112 | 81.4 (2.05) |
| | | | | (0.019) | (3.04) | (0.002) | |
| [symbol] a5 | 0.1 | Trs | 90.3 (1.56) | 0.223 | 36.2 | 0.138 | 88.4 (3.68) |
| | | | | (0.006) | (2.04) | (0.005) | |
| | 0.3 | Trs | 146 (1.29) | 0.256 | 35.7 | 0.118 | 75.9 (1.96) |
| | | | | (0.007) | (1.03) | (0.002) | |
| | 0.6 | Clr | 162 (1.73) | 0.221 | 36.7 | 0.109 | 69.8 (3.18) |
| | | | | (0.011) | (2.60) | (0.004) | |
| [symbol] Pa2.5 | 0.1 | Tbd/Ppt | 479 (202) | 0.428 | 31.9 | 0.141 | 89.1 (2.59) |
| | | | | (0.149) | (1.46) | (0.004) | |
| | 0.3 | Trs | 288 (106) | 0.416 | 33.6 | 0.151 | 95.3 (3.75) |
| | | | | (0.122) | (1.39) | (0.005) | |
| | 0.6 | Clr | 108 (1.16) | 0.317 | 33.2 | 0.162 | 102.5 (1.35) |
| | | | | (0.044) | (1.91) | (0.002) | |
| [symbol] Pa5 | 0.1 | Tbd/Ppt | 5810 (1480) | 0.436 | 17.9 | 0.134 | 88.5 (0.96) |
| | | | | (0.186) | (0.48) | (0.001) | |
| | 0.3 | Trs | 344 (199) | 0.3 | 34.6 | 0.138 | 91.1 (4.74) |
| | | | | (0.071) | (1.68) | (0.007) | |
| | 0.6 | Clr | 120 (1.62) | 0.283 | 30.3 | 0.146 | 96.6 (1.44) |
| | | | | (0.028) | (2.98) | (0.002) | |
| [symbol] Pa5 | 0.1 | Trs | 141 (21.8) | 0.226 | 34.3 | 0.140 | 91.6 (2.67) |
| | | | | (0.068) | (1.48) | (0.004) | |
| | 0.3 | Trs | 168 (4.10) | 0.275 | 33.5 | 0.124 | 81.2 (2.30) |
| | | | | (0.006) | (1.28) | (0.003) | |
| | 0.6 | Trs | 185 (4.23) | 0.270 | 33.0 | 0.118 | 77.0 (0.65) |
| | | | | (0.016) | (1.35) | (0.001) | |

*Form indicates physical appearance as Clear (Clr), Translucent (Trans), Turbid (Tbd), or precipitation (Ppt) [symbol]
indicates data missing or illegible when filed

The following Table 6 shows that the Zeta potential of quaternised copolymers roughly halved after addition of insulin, whereas the non-quaternised copolymers surface charges stayed roughly the same. This suggests that insulin was mostly confined within the hydrophobic core of the non-quaternised copolymer nanoparticles which increased hydrophobicity of the core and displaced some of the internal amine groups to the surface. In the case of the quaternised copolymers the loss of surface charge could be attributed to partial neutralisation of that charge by ionic interactions between the positively charged quaternised groups of the copolymer and the negatively charged insulin. TEM images supported this (see FIG. 8)

TABLE 6
Grafting (%) and properties of copolmers (n = 3, ±SD).

| | | | Physical | | | | |
|---|---|---|---|---|---|---|---|
| | | | appearance | | | | |
| | | | of 1 mg/mL | Hydro- | | | |
| | Hydro- | | solutions | dynamic | | | Mean Zeta |
| | phobic | | before | diameter, | | CAC, | Potential |
| Polymer | subst'n | Q, % | filtration | nm | PDI | mg/ml | mV |
| PAA | n/a | n/a | Clear | 1640 (484) | 0.95 | n/a | 22.6 |
| | | | | | (0.08) | | (1.04) |
| | | | | | 0.43 | | 31.7 |
| Ch2.5^{a} | 2.2 | n/a | Opaque | 230 (15) | (0.06) | 0.080 | (0.30) |
| | | | | | 0.41 | | 74.8 |
| QCh2.5^{a} | 2.2 | 68.0 | Translucent | 361 (37) | (0.07) | 0.080 | (1.10) |
| | | | | | 0.19 | | 46.9 |
| Ch5^{a} | 6.3 | n/a | Translucent | 167 (3) | (0.01) | 0.020 | (1.17) |
| | | | | | 0.26 | | 62.6 |
| QCh5^{a} | 6.3 | 71.0 | Clear | 115 (7) | (0.04) | 0.035 | (2.63) |
| | | | | | 0.27 | | 37.0 |
| Pa2.5 | 4.2 | n/a | Translucent | 205 (2) | (0.01) | 0.150 | (1.34) |
| | | | | | 0.60 | | 67.7 |
| QPa2.5 | 4.2 | 73.0 | Clear | 459 (117) | (0.04) | 0.180 | (1.24) |
| | | | | | 0.20 | | 44.0 |
| Pa5 | 6.6 | n/a | Translucent | 210 (2) | (0.008) | 0.080 | (2.18) |
| | | | | | 0.46 | | 54.6 |
| QPa5 | 6.6 | 65.0 | Clear | 316 (20) | (0.12) | 0.050 | (2.88) |
| | | | | | 0.60 | | 48.1 |
| Ce2.5 | 4 | n/a | Clear | 241 (25) | (0.05) | 0.160 | (3.52) |
| | | | | | 0.44 | | 57.8 |
| QCe2.5 | 4 | 78.0 | Clear | 353 (25) | (0.04) | 0.150 | (0.11) |
| | | | | | 0.535 | | 37.2 |
| Ce5 | 7.5 | n/a | Clear | 134 (6) | (0.09) | 0.110 | (2.30) |
| | | | | | 0.492 | | 48.5 |
| QCe5 | 7.5 | 70.5 | Clear | 441 (23) | (0.10) | 0.025 | (0.73) |

PAA = poly(allylamine);
CH = cholesterol;
Pa = palmitoyl;
Ce = cetyl;
Q = quaternised;
PDI = polydispersity index;
CAC = critical aggregate concentration.
^{a}Solutions filtered with 0.45 μm syringe filter before zeta potential and dynamic light scattering analysis.

Intracellular Localisation Study

The intracellular polymer uptake was studied using an experimental anticancer drug named PK4 (bis-naphthalimidopropyl spermine). A polymer-PK4 solution was used, consisting of QCPAA (0.1 μgml⁻¹) and drug (20 μM) in a medium containing 20% DMSO). Due to the inherent fluorescence of PK4, the uptake of PK4 into cells can be visualised using Fluorescence Microscopy. Caco-2 cells (human colorectal cancer cells) were maintained in Dulbecco's Modified Eagle's Medium (DMEM) supplemented with 10% foetal calf serum (FCS), 1% non-essential amino acids and 1% L-glutamine and 1% penicillin/streptomycin at 10% CO₂, 95% humidity and 37° C. Subsequently 2×10⁴ cells were seeded in 96-well microtitre plates and incubated for 24 h under the abovementioned conditions before addition of QCPAA polymer-PK4 solutions. The controls were 1) polymer only solutions (0.1 μml⁻¹), 2) PK4 only solutions (20 μM) and 3) medium. At specific time intervals the cells were photographed using a Leica DMRB fluorescence microscopy (Leica Microsystems, UK). The experiment above was repeated with the cells incubated at 4° C. instead of 37° C.

Unlike PK4, QCPAA did not possess inherent fluorescence and hence the fluorescence observed inside the cell was contributed by PK4 alone. For Caco-2 cells treated with PK4 (20 μM), fluorescence was not detected until 4.5 h indicating slow drug uptake by cells (FIG. 6). The QCPAA concentration used in this assay was 330 times less than the IC₅₀ of QCPAA (3 μgml⁻¹) determined by MTT assay after 24 h exposure to Caco-2 cells. When Caco-2 cells were incubated with QCPAA (0.1 μgml⁻¹) and PK4 (20 μM) solution, some fluorescence was observed after 30 min and the intensity increased hourly as shown in FIG. 6, demonstrating the ability of QCPAA to enhance the uptake of PK4 and increase the efficiency of this anticancer agent at the biocompatible concentration. When the above experiments were performed at 4° C., no fluorescence was detected which showed that QCPAA might utilise an active transport mechanism to facilitate drug uptake.

Advantages of the polymeric delivery species described herein include the fact that they can be produced at room temperature, using water and buffers which do not degrade the intended “passenger” entity to be delivered therein. This contrasts with work published by others relating to solid micro-/nanoparticles, block copolymers, and systems requiring use of high temperatures and organic solvents which are factors that would denature proteins thereby excluding a vast number of potentially valuable therapeutic agents required in modern medicine.

The polymers offer advantages with regard to hydrophobic entities, but also are useful in delivery of entities which have hydrophilic properties, and thus are universally applicable in drug delivery.

The invention by providing a hydrodynamic mixture of polymer and physiologically active entity, allows the polymers to encapsulate the entity in an aqueous medium by hydrophobic and ionic interactions to form nano-sized complexes which are readily delivered orally or parenterally. The resulting polymer complex is of nanoparticle range sizes with a T_{g} of less than 37° C. and deliverable in aqueous media as micelles of typically 100 to 500 nm in hydrodynamic diameter. When delivered orally, the polymer protects the entity from enzymes and pH changes during passage through the GI tract before it is released into a physiological circulation system.

Various modifications and variations to the described embodiments of the invention will be apparent to those skilled in the art without departing from the scope and the invention as described herein and defined in the claims. The word “comprising” as used herein is used in an inclusive sense. Although the invention has been described in connection with specific preferred embodiments, it should be understood that the invention as Claimed should not be unduly limited to such specific embodiments. Indeed, various modifications of the described modes of carrying out the invention which are obvious to those skilled by the present invention.

1. A polymer having a structure according to the following formula: 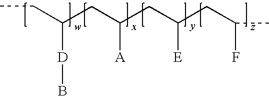
wherein:
A represents a hydrophilic group;
B represents a hydrophobic group;
D and E independently represent amine groups;
F represents an amine group substituted with a B group wherein the amine group is either substituted with an A group or the amine group is a tertiary amine;
where the molar ratio of monomeric unit Z to monomeric unit Y is 0:100 the molar ratio of monomeric unit W to monomeric unit Y is 0.01 to 100:100;
where the molar ratio of monomeric unit W to monomeric unit Y is 0:100 the molar ratio of monomeric unit Z to monomeric unit Y is 0.01 to 100:100;
the molar ratio of monomeric unit X to monomeric unit Y is 0 to 100:100.

2. The polymer claimed in claim 1, wherein the molar ratio of monomeric unit W to monomeric unit Y is 1 to 10:100.

3. The polymer claimed in claim 1, wherein the molar ratio of monomeric unit X to monomeric unit Y is 40 to 90:100.

4. The polymer claimed in claim 2, wherein the molar ratio of monomeric unit X to monomeric unit Y is 40 to 90:100.

5. The polymer claimed in claim 1, wherein the molar ratio of monomeric unit Z to monomeric unit Y is 1 to 10:100.

6. The polymer claimed in claim 2, wherein the molar ratio of monomeric unit Z to monomeric unit Y is 1 to 10:100.

7. The polymer claimed in claim 3, wherein the molar ratio of monomeric unit Z to monomeric unit Y is 1 to 10:100.

8. The polymer claimed in claim 4, wherein the molar ratio of monomeric unit Z to monomeric unit Y is 1 to 10:100.

9. The polymer monomeric unit W and monomeric unit Z wherein the molar ratio of monomeric unit W to monomeric unit Y is 10 to 20:100 and the molar ratio of monomeric unit Z to monomeric unit Y is 1 to 5:100.

10. The polymer claimed in claim 1, wherein the hydrophobic group B is a hydrocarbon chain having a carbon backbone of 8 to 24 carbon atoms.

11. The polymer The polymer claimed in claim 10, wherein the carbon backbone comprises alkyl and aryl components.

12. The polymer claimed in claim 1, wherein the hydrophobic group B is selected from the group consisting of saturated and unsaturated hydrocarbon chains substituted with one or more substituents, the said substituents being selected from the group consisting of ester, aldehyde, ketone, amine, amide, alkenyl, alkynyl, acyl, hydroxyl alkyl, hydroxy acyl and sugar groups.

13. The polymer claimed in claim 1, wherein the hydrophobic group B is a cholesterol-based group having the structure as shown below:

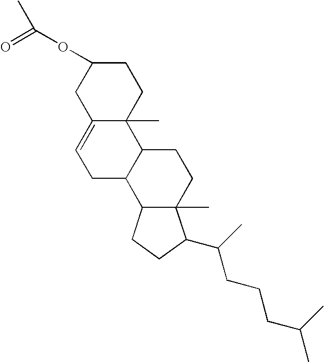

14. The polymer claimed in claim 1, wherein the hydrophobic group B is a palmitoyl based soup.

15. The polymer claimed in claim 1, wherein the hydrophobic group B is a cetyl based group.

16. The polymer claimed in claim 1, wherein the hydrophilic group A is selected from the group consisting of a primary amine substituted with a hydrophilic group, a secondary amine substituted with a hydrophilic group and a tertiary amine group.

17. The polymer claimed in claim 1, wherein the hydrophilic group A is selected from the group consisting of a substituted primary amine, a substituted secondary amine and a substituted tertiary amine, wherein the substitution on any of said substituted primary, secondary and tertiary amines, is selected from the group consisting of one or more hydrogen, alkyl, alkenyl, alkynyl, aryl, acyl, hydroxyl alkyl, hydroxyl acyl, polyethylene glycol, methyl glycolate and sugar groups.

18. The polymer of claim 17 wherein the hydrophilic group A is a quaternary ammonium moiety substituted with one or more groups selected from —CH₃, —H, groups having 1 to 6 carbon atoms, and sugar groups.

19. The polymer claimed in claim 1, wherein D and E independently represent —CH₂NH₂ or —CH₂NHR where R represents an hydrophobic group B.

20. The polymer claimed in claim 1, wherein D and E independently represent —CH₂NH₂ or —CH₂NHR where R represents a substituted or unsubstituted hydrocarbon chain.

21. The polymer claimed in claim 1 having the structure shown below: 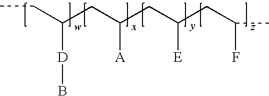
where:
D suitably represents CH₂—NH;
A suitably represents CH₂—N-hydrophilic group;
E suitably represents CH₂—NH₂;
F suitably represents

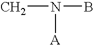

22. The polymer claimed in claim 1, wherein said polymer is a based upon a polyallylamine (PAA) polymer which has an average molecular weight of about 10 to 25 kD.

23. A composition of matter comprising the polymer claimed in claim 1, and a pharmaceutically acceptable vehicle.

24. The composition of matter claimed in claim 23, wherein the pharmaceutically acceptable vehicle is an aqueous solution.

25. The composition of matter claimed in claim 23, wherein the pharmaceutically acceptable vehicle is an emulsion.

26. The composition of matter claimed in claim 23, wherein the pharmaceutically acceptable vehicle is a suspension.

27. The composition of matter claimed in claim 23, wherein the pharmaceutically acceptable vehicle is a non-aqueous solution.

28. The composition of matter claimed in claim 23, wherein the pharmaceutically acceptable vehicle is selected from the group consisting of a solvent, an oil, and an organic ester.

29. The composition of matter claimed in claim 23, wherein the ratio of polymer to pharmaceutically acceptable vehicle is 0.001 to 10:1.

30. A delivery composition comprising the composition of matter claimed in claim 23, and an entity to be delivered, said entity having a limited solubility in a media.

31. The delivery composition claimed in claim 30, wherein the entity has a limited aqueous solubility.

32. The delivery composition claimed in claim 31, wherein the entity has an aqueous solubility of 0.001 to 0.2 mg/ml at a temperature of 15 to 25° C.

33. The delivery composition claimed in claim 30, wherein the entity is a drug.

34. The delivery composition claimed in claim 30, wherein the entity is a peptide.

35. The delivery composition claimed in claim 30, wherein the entity is a protein.

36. The delivery composition claimed in claim 30, wherein the entity is a polynucleotide.

37. The delivery composition claimed in claim 30, wherein the entity is selected from the group consisting of prednisolone, oestradiol, testosterone, griseofulvin, propofol, bis-naphthalimidopropyl spermine, cyclosporin, insulin and glucagon.

38. The delivery composition claimed in claim 30, wherein the ratio of entity to polymer by weight is 10 to 90:200.

39. The delivery composition claimed in claim 30, presented in a pharmaceutical form that is adapted for oral delivery.

40. The delivery composition claimed in claim 30, presented in a pharmaceutical form that is adapted for parenteral delivery.

41. The delivery composition claimed in claim 30, prepared as a medicament for use in therapy.

42. A method of medical treatment comprising the step of administering the delivery composition claimed in claim 30, to a patient in need thereof.

43. A micelle-containing system for delivering a therapeutic entity, wherein a micelle comprises the polymer claimed in claim 1, and wherein at least one therapeutic entity is releasably contained within the said micelle.

44. The micelle-containing system claimed in claim 43, wherein the therapeutic entity is selected from the group consisting of a drug, peptide, protein, and a polynucleotide.

45. A method of increasing the solubility of an entity having limited solubility in a media comprising the steps of mixing the entity, the polymer claimed in claim 1 and the media together to form a solution.

46. The method claimed in claim 45, wherein the polymer is mixed with the media prior to mixing with the entity.

47. The method claimed in claim 45, wherein the polymer is mixed with entity the prior to mixing with the media.

48. The method claimed in claim 45, wherein the ratio of polymer to entity mixed is 10 to 90:200 by weight.

49. The method claimed in claim 45, wherein the solubility is increased such that absorption of the entity by a physiological system of the human or animal body is promoted.

50. The method claimed in claim 45, wherein the ability of the entity to cross cell barriers is maximised and the uptake of the entity in cells is facilitated.
